(19) United States
(12) Patent Application Publication
(10) Pub. No.: US 2016/0201247 A1
(43) Pub. Date: Jul. 14, 2016

(54) WASHING MACHINE APPLIANCE

(71) Applicant: General Electric Company, Schenectady, NY (US)

(72) Inventors: Stephen Edward Hettinger, Louisville, KY (US); John Joseph Roetker, Louisville, KY (US)

(21) Appl. No.: 14/592,979

(22) Filed: Jan. 9, 2015

(51) Int. Cl. D06F 39/00; D06F 39/08; A61L 2/18; D06F 33/02

(52) U.S. Cl. CPC D06F 39/005; D06F 33/02; D06F 39/087; D06F 39/083; A61L 2/18; D06F 2232/04; D06F 2232/06; D06F 2202/085; D06F 2204/08; D06F 2204/02

(57) ABSTRACT

A method of sanitizing articles in a wash chamber of a washing machine appliance is provided. The method includes providing the wash chamber with an oxygen bleach wash additive and, optionally, with a detergent wash additive, determining a water fill volume target, and adding a first wash volume of water to the wash chamber. The first wash volume is determined based on a fixed percentage of the water fill volume target in is less than the water fill volume target such that the oxygen bleach wash additive is present in a relatively concentrated amount.

### FIELD OF THE INVENTION

The present subject matter relates generally to washing machine appliances and methods for operating the same.

### BACKGROUND OF THE INVENTION

Washing machine appliances generally include a tub for containing wash fluid, e.g., water and detergent, bleach and/or other wash additives. A basket is rotatably mounted within the tub and defines a wash chamber for receipt of articles for washing. During normal operation of such washing machine appliances, wash fluid is directed into the tub and onto articles within the wash chamber of the basket. The basket or an agitation element can rotate at various speeds to agitate articles within the wash chamber in the wash fluid, to wring wash fluid from articles within the wash chamber, etc.

Certain washing machine appliances are additionally capable of performing sanitation wash cycles to clean and disinfect articles within the wash chamber of the basket. The sanitation wash cycles require an in-tub heater to raise a temperature of the wash fluid/water within the wash chamber of the basket above a certain threshold to provide the desired cleaning and disinfecting functionalities. However, such in-tub heaters are additional to the standard washing machine appliance components, and thus require additional expenditures in order to incorporate. Additionally, washing machine appliances using in-tub heaters for a sanitation cycle can increase a total cycle time as the in-tub heater must heat the water to the desired temperature.

Accordingly, improved washing machine appliances and methods for operating washing machine appliances are desired in the art. In particular, washing machine appliances and methods capable of providing sanitation cycles without an in-tub heater would be useful.

### BRIEF DESCRIPTION OF THE INVENTION

Aspects and advantages of the invention will be set forth in part in the following description, or may be apparent from the description, or may be learned through practice of the invention.

In a first exemplary aspect, a method of sanitizing articles in a wash chamber of a washing machine appliance is provided. The method includes initiating a sanitation wash cycle; providing the wash chamber of the washing machine appliance with a wash additive, the wash additive including an oxygen bleach; and determining a water fill volume target. The method also includes determining a first wash volume based on a fixed percentage of the water fill volume target. The first wash volume is less than the water fill volume target. The method also includes adding the first wash volume of water to the wash chamber and initiating a secondary operation. Additionally, the method includes adding a second wash volume of water, the sum of the first wash volume and second wash volume being substantially equal to the water fill volume target.

In a second exemplary embodiment, a washing machine appliance is provided. The washing machine appliance includes a tub and a basket positioned within the tub. The basket is rotatable within the tub and defines a wash chamber for receipt of articles for washing. The washing machine appliance additionally includes a flow regulator and a controller in operable communication with the flow regulator. The controller is configured to execute a sanitation cycle including determining a water fill volume target and determining a first wash volume based on a fixed percentage of the water fill volume target. The first wash volume is less than the determined water fill volume target. The sanitation cycle additionally includes adding the first wash volume of water to the wash chamber, initiating a secondary operation, and adding a second wash volume of water. The sum of the first wash volume and second wash volume is substantially equal to the water fill volume target.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures.

FIG. 1 provides a perspective view of a washing machine appliance according to an exemplary embodiment of the present subject matter.

FIG. 2 provides a front, section view of the exemplary washing machine appliance of FIG. 1.

FIG. 3 provides a flow chart of a method in accordance with an exemplary aspect of the present disclosure.

FIG. 4 provides a chart of certain variables from a washing machine appliance executing a sanitation wash cycle in accordance with an exemplary aspect of the present disclosure.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

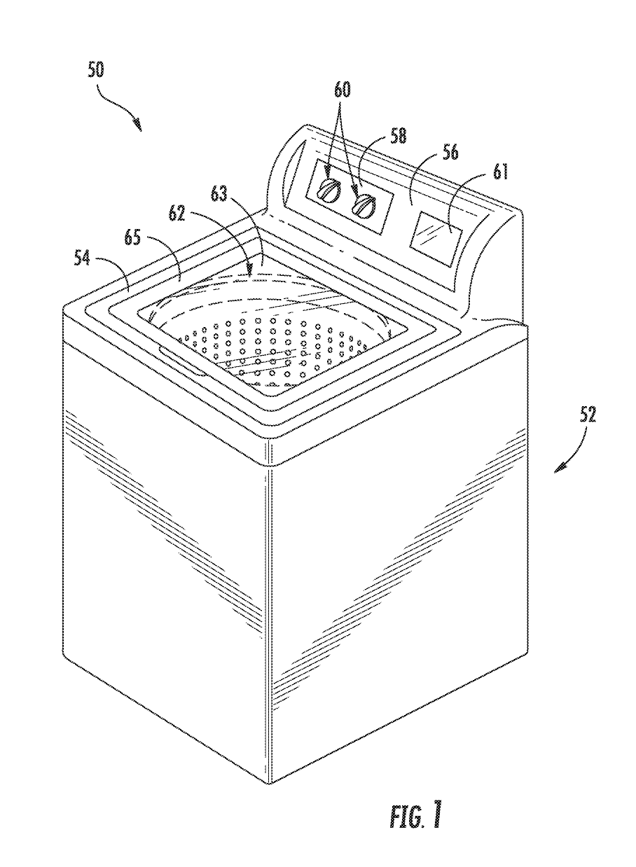

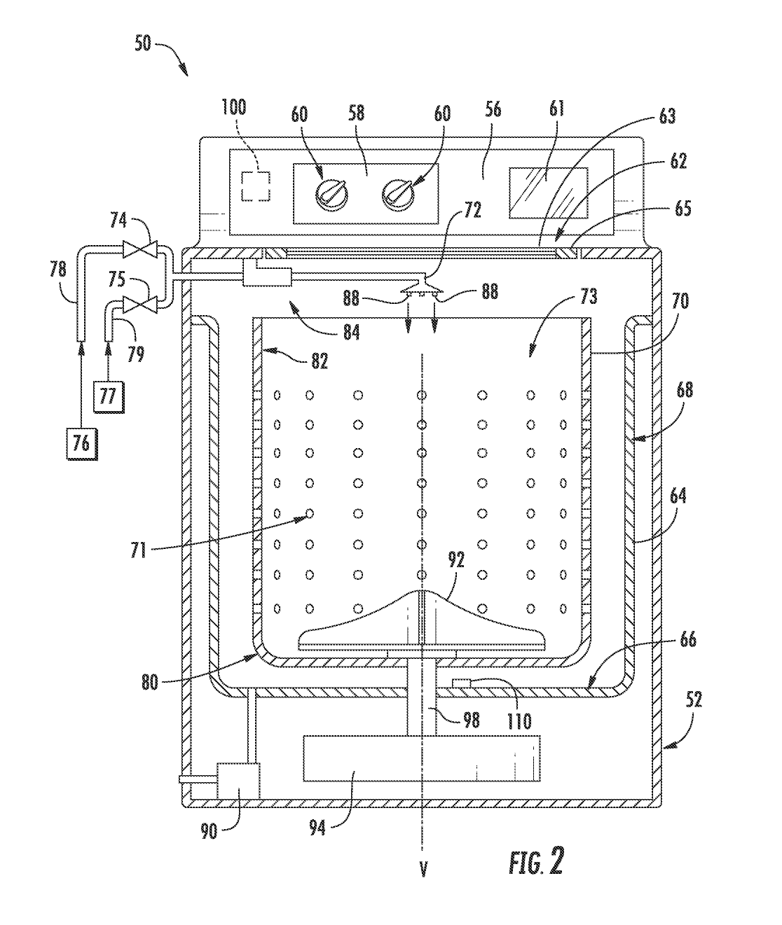

FIG. 1 is a perspective view of a washing machine appliance 50 according to an exemplary embodiment of the present subject matter. As may be seen in FIG. 1, washing machine appliance 50 includes a cabinet 52 and a cover 54. A backsplash 56 extends from cover 54, and a control panel 58 including a plurality of input selectors 60 is coupled to backsplash 56. Control panel 58 and input selectors 60 collectively form a user interface input for operator selection of machine cycles and features, and in one embodiment, a display 61 indicates selected features, a countdown timer, and/or other items of interest to machine users. A lid 62 is mounted to cover 54 and is rotatable between an open position (not shown) facilitating access to a wash tub 64 (FIG. 2) located within cabinet 52 and a closed position (shown in FIG. 1) forming an enclosure over tub 64.

Lid 62 in exemplary embodiment includes a transparent panel 63, which may be formed of, for example, glass, plastic, or any other suitable material. The transparency of the panel 63 allows users to see through the panel 63, and into the tub 64 when the lid 62 is in the closed position. In some embodiments, the panel 63 may itself generally form the lid 62. In other embodiments, the lid 62 may include the panel 63 and a frame 65 surrounding and encasing the panel 63. Alternatively, panel 63 need not be transparent.

FIG. 2 provides a front, cross-section view of the exemplary washing machine appliance 50 of FIG. 1. As may be seen in FIG. 2, tub 64 includes a bottom wall 66 and a sidewall 68. A wash drum or wash basket 70 is rotatably mounted within tub 64. In particular, basket 70 is rotatable about a vertical axis V. Thus, washing machine appliance is generally referred to as a vertical axis washing machine appliance. Basket 70 defines a wash chamber 73 for receipt of articles for washing and extends, e.g., vertically, between a bottom portion 80 and a top portion 82. Basket 70 includes a plurality of openings or perforations 71 therein to facilitate fluid communication between an interior of basket 70 and tub 64.

A nozzle 72 is configured for flowing a liquid into tub 64. In particular, nozzle 72 may be positioned at or adjacent top portion 82 of basket 70. Nozzle 72 may be in fluid communication with one or more water sources 76, 77 in order to direct liquid (e.g. water) into tub 64 and/or onto articles within chamber 73 of basket 70. Nozzle 72 may further include apertures 88 through which water may be sprayed into the tub 64. Apertures 88 may, for example, be tubes extending from the nozzles 72 as illustrated, or simply holes defined in the nozzles 72 or any other suitable openings through which water may be sprayed. Nozzle 72 may additionally include other openings, holes, etc. (not shown) through which water may be flowed, i.e. sprayed or poured, into the tub 64.

Various valves may regulate the flow of fluid through nozzle 72. For example, a flow regulator may be provided to control a flow of hot and/or cold water into the wash chamber of washing machine appliance 50. For the embodiment depicted, the flow regulator includes a hot water valve 74 and a cold water valve 75. The hot and cold water valves 74, 75 are utilized to flow hot water and cold water, respectively, therethrough. Each valve 74, 75 can selectively adjust to a closed position in order to terminate or obstruct the flow of fluid therethrough to nozzle 72. The hot water valve 74 may be in fluid communication with a hot water source 76, which may be external to the washing machine appliance 50. The cold water valve 75 may be in fluid communication with a cold water source 77, which may be external to the washing machine appliance 50. The cold water source 77 may, for example, be a commercial water supply, while the hot water source 76 may be, for example, a water heater. Such water sources 76, 77 may supply water to the appliance 50 through the respective valves 74, 75. A hot water conduit 78 and a cold water conduit 79 may supply hot and cold water, respectively, from the sources 76, 77 through the respective valves 74, 75 and to the nozzle 72.

An additive dispenser 84 may additionally be provided for directing a wash additive, such as detergent, bleach, liquid fabric softener, etc., into the tub 64. More particularly, as will be discussed below, additive dispenser 84 may be provided to direct an oxygen bleach wash additive and detergent wash additive into the tub 64. For example, dispenser 84 may be in fluid communication with nozzle 72 such that water flowing through nozzle 72 flows through dispenser 84, mixing with wash additive at a desired time during operation to form a liquid or wash fluid, before being flowed into tub 64. For the embodiment depicted, nozzle 72 is a separate downstream component from dispenser 84. In other exemplary embodiments, however, nozzle 72 and dispenser 84 may be integral, with a portion of dispenser 84 serving as the nozzle 72, or alternatively dispenser 84 may be in fluid communication with only one of hot water valve 74 or cold water valve 75. In still other exemplary embodiments, the washing machine appliance 50 may not include a dispenser, in which case a user may add one or more wash additives directly to wash chamber 73. A pump assembly 90 (shown schematically in FIG. 2) is located beneath tub 64 and basket 70 for gravity assisted flow to drain tub 64.

An agitation element 92, shown as an impeller in FIG. 2, may be disposed in basket 70 to impart an oscillatory motion to articles and liquid in chamber 73 of basket 70. In various exemplary embodiments, agitation element 92 includes a single action element (i.e., oscillatory only), double action (oscillatory movement at one end, single direction rotation at the other end) or triple action (oscillatory movement plus single direction rotation at one end, singe direction rotation at the other end). As illustrated in FIG. 2, agitation element 92 is oriented to rotate about vertical axis V. Alternatively, basket 70 may provide such agitating movement, and agitation element 92 is not required. Basket 70 and agitation element 92 are driven by a motor 94, such as a pancake motor. As motor output shaft 98 is rotated, basket 70 and agitation element 92 are operated for rotatable movement within tub 64, e.g., about vertical axis V. It should be appreciated, however, that in other exemplary embodiments, the exemplary washing machine appliance 50 may not include an agitation element 92, and instead wash machine appliance 50 may agitate articles positioned within wash chamber 73 by, e.g., rotating basket 70. Washing machine appliance 50 may also include a brake assembly (not shown) selectively applied or released for respectively maintaining basket 70 in a stationary position within tub 64 or for allowing basket 70 to spin within tub 64.

Various sensors may additionally be included in the washing machine appliance 50. For example, a pressure sensor 110 may be positioned in the tub 64 as illustrated or, alternatively, may be remotely mounted in another location within the appliance 50 and be operationally connected to tub 64 by a hose (not shown). Any suitable pressure sensor 110, such as an electronic sensor, a manometer, or another suitable gauge or sensor, may be utilized. The pressure sensor 110 may generally measure the pressure of water in the tub 64. This pressure can then be utilized to estimate the height or level of water in the tub 64. Additionally, a suitable speed sensor can be connected to the motor 94, such as to the output shaft 98 thereof, to measure speed and indicate operation of the motor 94. Other suitable sensors, such as temperature sensors, etc., may additionally be provided in the washing machine appliance 50.

Operation of washing machine appliance 50 is controlled by a processing device or controller 100, that is operatively coupled to the input selectors 60 located on washing machine backsplash 56 (shown in FIG. 1) for user manipulation to select washing machine cycles and features. Controller 100 may further be operatively coupled to various other components of appliance 50, such as the flow regulator (including valves 74, 75), motor 94, pressure sensor 110, and other suitable sensors, etc. In response to user manipulation of the input selectors 60, controller 100 may operate the various components of washing machine appliance 50 to execute selected machine cycles and features.

Controller 100 may include a memory and microprocessor, such as a general or special purpose microprocessor operable to execute programming instructions or micro-control code associated with a cleaning cycle. The memory may represent random access memory such as DRAM, or read only memory such as ROM or FLASH. In one embodiment, the processor executes programming instructions stored in memory. The memory may be a separate component from the processor or may be included onboard within the processor. Alternatively, controller 100 may be constructed without using a microprocessor, e.g., using a combination of discrete analog and/or digital logic circuitry (such as switches, amplifiers, integrators, comparators, flip-flops, AND gates, and the like) to perform control functionality instead of relying upon software. Control panel 58 and other components of washing machine appliance 50 may be in communication with controller 100 via one or more signal lines or shared communication busses.

While described in the context of specific embodiments of washing machine appliance 50, using the teachings disclosed herein it will be understood that washing machine appliance 50 is provided by way of example only. Other washing machine appliances having different configurations (such as horizontal-axis washing machine appliances), different appearances, and/or different features may also be utilized with the present subject matter as well.

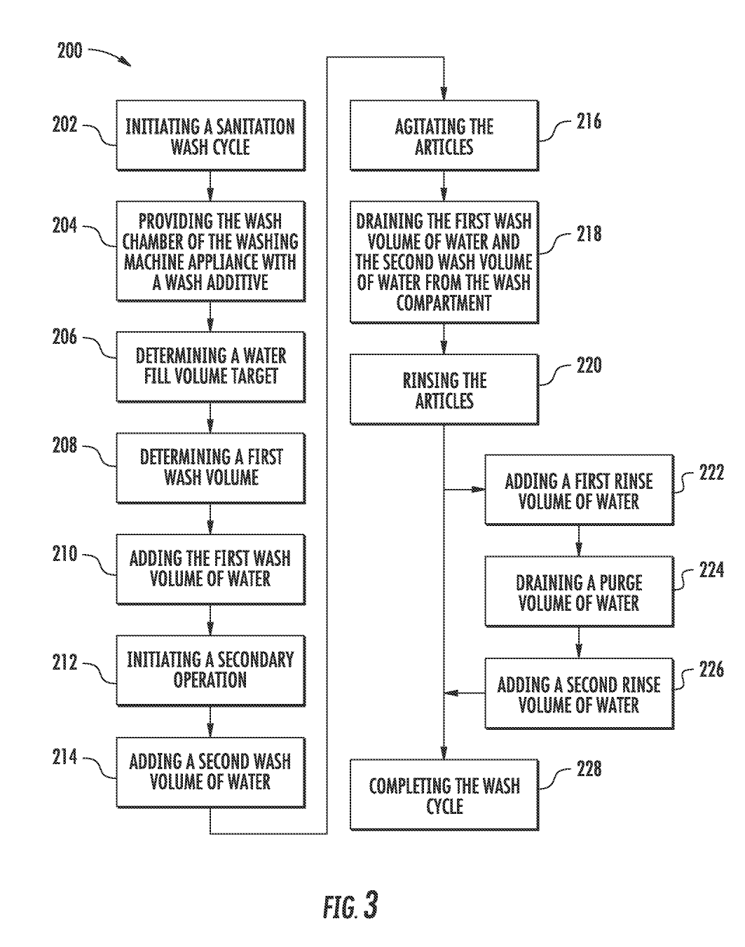

Referring now to FIG. 3, a flow chart of a method (200) for operating a washing machine appliance in accordance with an exemplary aspect of the present disclosure is provided. More particularly, FIG. 3 provides a flow chart of an exemplary method (200) for sanitizing articles in a wash chamber of a washing machine appliance. The method (200) may be performed by exemplary washing machine appliance 50 depicted in FIGS. 1 and 2, including controller 100 of washing machine appliance 50. Accordingly, controller 100 may be configured to execute a sanitation wash cycle described below with reference to FIG. 3.

The exemplary method (200) of FIG. 3 includes at (202) initiating a sanitation wash cycle. Initiating the sanitation wash cycle at (202) may include, for example, loading one or more articles for washing (e.g., clothing, towels, etc.) into the wash chamber of the washing machine appliance and selecting a sanitation wash cycle through, e.g., manipulation of one or more control input selectors. The exemplary method (200) further includes at (204) providing the wash chamber of the washing machine appliance with a wash additive, the wash additive including an oxygen bleach wash additive. In certain embodiments, the wash additive may additionally include a detergent additive. As used herein, “oxygen bleach wash additive” may refer to any wash additive including sodium percarbonate or peroxide chemistry. Moreover, it has been determined that oxygen bleach wash additives may sanitize more effectively when combined with hot water. Accordingly, the wash additive may be referred to as a temperature activated wash additive defining an activation temperature. In certain exemplary embodiments, the activation temperature may be below a typical water temperature provided by a hot water tap, or hot water valve, to the washing machine appliance. Activators, such as TAED or NOBS, may also be included ingredients in the oxygen bleach wash additive. Such activators may lower the reaction temperature in the wash solution during a sanitation wash cycle.

The wash additive may be provided at (204) in any suitable manner. For example, in certain exemplary aspects, the wash additive may be provided at (204) through a dispenser assembly, such as dispenser 84 described above, or alternatively may be provided at (204) directly to the wash chamber by, e.g., a user.

In certain exemplary aspects, the method of FIG. 3 may further include providing the wash chamber of the washing machine appliance with wash additives other than oxygen bleach wash additive. For example, in other exemplary aspects, the method may additionally include providing bleach, fabric softener, etc. to the wash chamber of the washing machine appliance.

Referring still to FIG. 3, the exemplary method (200) further includes at (206) determining a water fill volume target. The water fill volume target may be determined at (206) based on, e.g., a weight of the articles positioned in the wash chamber, absorption characteristics of the articles positioned in the wash chamber, and any other factors that may affect how much water should be used for washing operations. A first wash volume is then determined at (208) based on a fixed percentage of the water fill volume target. The first wash volume is less than the water fill volume target. For example, the first wash volume may be low enough to ensure the oxygen bleach wash additive is not unnecessarily diluted (i.e., maintained at a relatively high concentration), but high enough to effectively saturate each of the articles positioned within the wash chamber of the washing machine appliance. In certain exemplary aspects, the first wash volume may be between about forty percent (40%) and about eighty percent (80%) of the water fill volume target. However, in other exemplary aspects, the first wash volume may be between about fifty percent (50%) and about seventy percent (70%) of the water fill volume target. In still other exemplary aspects, the first wash volume may be about sixty percent (60%) of the water fill volume target. Notably, as used herein, terms of approximation, such as “about,” “substantially,” and “approximately,” refer to being within a ten percent (10%) margin of error.

The exemplary method (200) of FIG. 3 further includes at (210) adding the first wash volume of water to the wash chamber of the washing machine appliance. In certain exemplary aspects, adding the first wash volume of water to the wash chamber at (210) may include adding the first wash volume of water substantially from a hot water tap, or hot water valve, of a flow regulator of the washing machine appliance. Notably, adding the first wash volume of water substantially from a hot water tap may include adding a nominal amount of water from a cold water tap, e.g., if required for dispensing of the wash additive. The first wash volume of water may accordingly define a temperature greater than the activation temperature of the oxygen bleach wash additive, as discussed above. It should be appreciated, however, that adding the first wash volume of water to the wash chamber at (210) may additionally, or alternatively, include adding the first wash volume of water from a cold water tap, or cold water valve.

After the first wash volume of water is added at (210), the method (200) further includes at (212) initiating a secondary operation. In certain exemplary aspects, the secondary operation initiated at (212) includes agitating the articles positioned within the wash chamber of the washing machine appliance for a determined time. In other exemplary aspects, the secondary operation initiated at (212) may additionally, or alternatively, include soaking the articles positioned within the wash chamber of the washing machine appliance for a determined time period.

A method (200) in accordance with such an exemplary aspect may effectively sanitize articles positioned within the wash chamber of the washing machine appliance without the use of, e.g., a secondary heater. More particularly, a method (200) in accordance with such an exemplary aspect may sanitize articles positioned within the wash chamber by combining an oxygen bleach wash additive with a first wash volume of water at an elevated temperature, such that a relatively concentrated amount of oxygen bleach wash additive is activated by the first wash volume of water to sanitize the articles.

The method (200) of FIG. 3 further includes at (214) adding a second wash volume of water. The sum of the first wash volume and the second wash volume are substantially equal to the water fill volume target. Accordingly, after adding the second wash volume of water at (214) to the wash chamber of the washing machine appliance, an amount of water may be present in the wash chamber equal to the water fill volume target. Moreover, after adding the second wash volume of water at (214), the method (200) of FIG. 3 further includes initiating additional wash operations. For example, the method of FIG. 3 includes at (216) agitating the articles positioned within the wash chamber subsequent to adding the second wash volume of water at (214).

For the exemplary aspect depicted, concluding the additional wash operations also concludes a sanitizing/washing phase of the sanitation wash cycle. Once the washing of the articles position within the wash chamber is complete, i.e., once the sanitizing/washing phase of the sanitation wash cycle is complete, the method (200) further includes at (218) draining the first wash volume of water and the second wash volume of water from the wash chamber, and at (220) rinsing the articles positioned within the wash chamber. For the exemplary aspect of FIG. 3, rinsing the articles at (220) positioned within the wash chamber includes at (222) adding a first rinse volume of water to the wash chamber, at (224) draining a purge volume of water from the wash chamber, and subsequently at (226) adding a second rinse volume of water to the wash chamber. The first rinse volume is less than the water fill volume target. For example, the first rinse volume may be less than about three gallons of water. However, in other exemplary aspects, the first rinse volume may be less than about two gallons of water, or further may be less than about one gallon of water.

Additionally, the purge volume may be less than the first rinse volume, or alternatively may be equal to the first rinse volume. The sum of the first rinse volume, the purge volume, and the second rinse volume may be equal to or greater than the water fill volume target. Notably, in certain exemplary aspects, adding the first rinse volume of water at (222) and draining the purge volume of water at (224) may occur simultaneously, or alternatively, may occur sequentially. Further, adding the second rinse volume of water at (226) may include adding the second rinse volume of water directly after adding the first rinse volume of water at (222), or alternatively may include adding the second rinse volume of water after draining the purge volume of water at (224).

A method (200) in accordance with such an exemplary aspect may allow for an effective “single fill” rinse of the articles positioned within the wash chamber. More particularly, the partial rinse at (222) and purge at (224) of such a method (200) may effectively remove any remaining wash additive residue positioned within, e.g., the wash chamber, the tub, and/or a sump area of the washing machine appliance, such that a second full rinse may not be required after draining the first and second rinse volumes of water from the wash chamber at (218). Accordingly, a method (200) in accordance with such an exemplary aspect, may allow for a more energy-efficient wash cycle.

Subsequent to rinsing the articles positioned within the wash chamber of the washing machine appliance at (220), the method (200) may further include spinning the wash basket to wring out the articles positioned within the wash chamber (not shown) and at (228) completing the sanitation wash cycle. It should be appreciated, however, that in other exemplary aspects, the method (200) of FIG. 3 additionally include any other suitable steps or processes to assist in washing and/or sanitizing the articles positioned within the wash chamber.

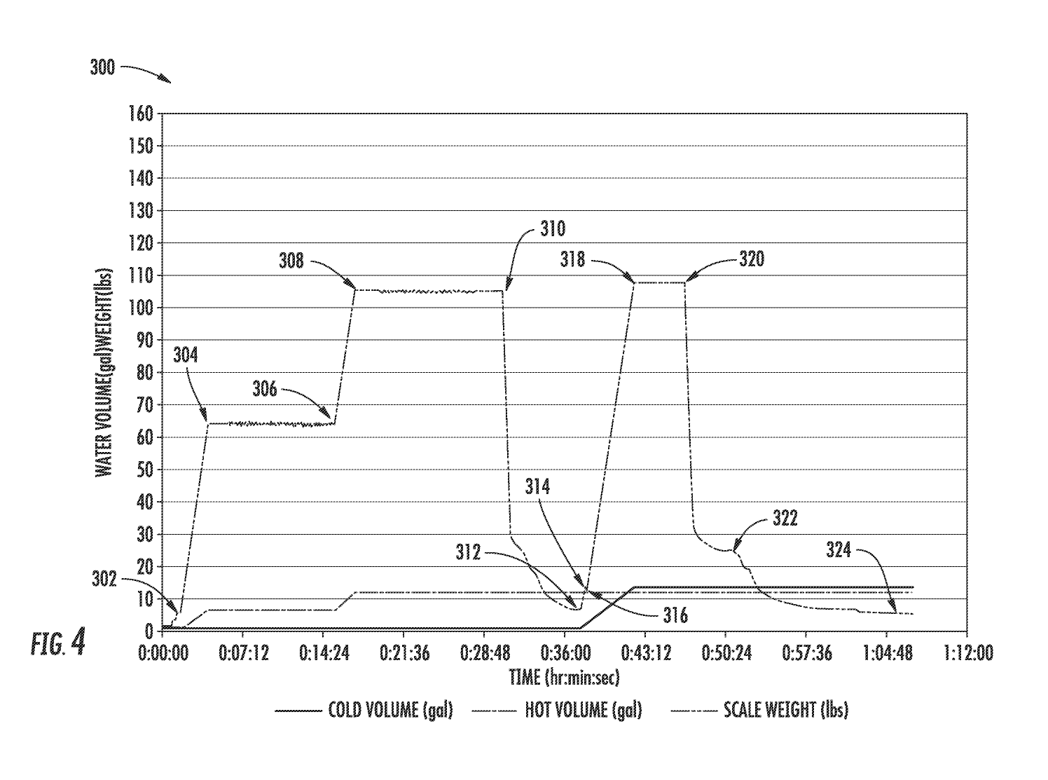

Referring now to FIG. 4, a chart 300 is provided depicting a weight of a wash chamber of a washing machine appliance, a volume of hot water added to a wash chamber of the washing machine appliance, and a volume of cold water added to the wash chamber of the wash machine appliance during a sanitation wash cycle in accordance with an exemplary aspect of the present disclosure. For example, the chart 300 provided in FIG. 4 may depict certain variables of a washing machine appliance during a sanitation wash cycle operated in accordance with the exemplary method of FIG. 3.

The chart 300 of FIG. 4 depicts the exemplary sanitation wash cycle starting at 302. At such time, a first wash volume of water is provided substantially from a hot water tap or valve. More particularly, for the exemplary sanitation wash cycle charted in FIG. 4, the first wash volume of water is about seven gallons of hot water and about one gallon of cold water. The cold water may be dispensed in order to provide a wash chamber of the washing machine appliance with an oxygen bleach wash additive and, optionally a detergent wash additive. For example, the cold water may be routed through, e.g., a dispenser assembly of the washing machine appliance to provide the wash chamber of the washing machine appliance with the wash additive(s), including an oxygen bleach wash additive, and optionally a detergent wash additive.

Starting at 304, after the first wash volume of water has been added, the exemplary sanitation wash cycle includes initiating a series of secondary operations. More particularly, the exemplary sanitation wash cycle initiates at 304 a series of agitation modes, wherein the articles positioned within the wash chamber are agitated, and soak modes, wherein the articles positioned within the wash chamber are soaked. As discussed, such operations may sanitize any articles positioned within the wash chamber of the wash machine appliance.

Once the secondary operations complete, at 306, the sanitation wash cycle includes adding a second wash volume of water. As indicated, for the exemplary embodiment depicted, the second wash volume of water is approximately five additional gallons of hot water. At 308, once the addition of the second wash volume of water is complete, the sanitation wash cycle may include initiating another series of, e.g., agitation modes and/or soak modes to complete the washing operations. Subsequently, at 310, the sanitation wash cycle includes draining the first wash volume of hot water and the second wash volume of hot water from the wash chamber of the washing machine appliance.

At 312, the sanitation wash cycle includes initiating a rinsing operation. More particularly, for the embodiment depicted, the sanitation wash cycle includes adding a first rinse volume of water to wash chamber at 312. For the embodiment depicted, the first rinse volume of water is approximately one gallon of water. The sanitation wash cycle subsequently includes, at 314 draining a purge volume of water from the wash chamber. For the embodiment depicted, the purge volume of water is less than the first rinse volume of water. Such a step may effectively remove any wash additive residue left over from washing operations, such that sanitation wash cycle may effectively rinse the articles positioned within the wash chamber using a “single fill.” Subsequent to draining the purge volume of water, at 316, the sanitation wash cycle includes adding a second rinse volume of water to the wash chamber. As shown, the sum of the first rinse volume, the purge volume, and the second rinse volume is greater than the water fill volume target of the washing machine appliance.

At 318, once the second rinse volume of water has been added, the sanitation wash cycle includes soaking the articles positioned within the wash chamber for a predetermined amount of time. Notably, however, in other exemplary embodiments, the sanitation wash cycle may additionally include, e.g., one or more agitation modes along with the soak mode depicted. Subsequently, at 320, the sanitation wash cycle includes draining the remaining portion of the first rinse volume of water as well as the second rinse volume of water from the wash chamber, and at 322, initiating a spin operation to wring remaining water out of the articles positioned within the wash chamber. The sanitation wash cycle then completes at 324.

It should be appreciated, however, that the exemplary sanitation wash cycle charted in FIG. 4 is by way of example only. In other exemplary embodiments, the wash cycle may include any other suitable time frames, wash volumes, drain volumes, secondary operations, etc.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A method of sanitizing articles in a wash chamber of a washing machine appliance, the method comprising:
initiating a sanitation wash cycle;
providing the wash chamber of the washing machine appliance with a wash additive, the wash additive including an oxygen bleach;
determining a water fill volume target;
determining a first wash volume based on a fixed percentage of the water fill volume target, the first wash volume being less than the water fill volume target;
adding the first wash volume of water to the wash chamber;
initiating a secondary operation; and
adding a second wash volume of water, the sum of the first wash volume and second wash volume being substantially equal to the water fill volume target.

2. The method of claim 1, wherein the first wash volume is between about forty percent and about eighty percent of the water fill volume target.

3. The method of claim 1, wherein adding the first wash volume of water to the wash chamber comprises adding the first wash volume of water substantially from a hot water tap, and wherein the wash additive is a temperature activated wash additive.

4. The method of claim 3, wherein the temperature activated wash additive defines an activation temperature, and wherein the first wash volume of water defines a temperature greater than or equal to the activation temperature.

5. The method of claim 1, wherein the secondary operation comprises agitating the articles positioned within the wash chamber for a determined time period and soaking the articles positioned within the wash chamber for a determined time period.

6. The method of claim 5, wherein providing the wash chamber of the washing machine appliance with the wash additive comprises adding the wash additive directly to the wash chamber prior to initiating the sanitation wash cycle.

7. The method of claim 1, further comprising
agitating the articles positioned within the wash chamber subsequent to adding the second wash volume of water.

8. The method of claim 1, further comprising
draining the first wash volume of water and the second wash volume of water from the wash chamber; and
rinsing the articles positioned within the wash chamber.

9. The method of claim 8, wherein rinsing the articles positioned within the wash chamber further comprises
adding a first rinse volume of water to the wash chamber;
draining a purge volume of water from the wash chamber; and
adding a second rinse volume of water to the wash chamber.

10. The method of claim 9, wherein the first rinse volume is less than about three gallons of water.

11. The method of claim 9, wherein the first rinse volume is less than the water fill volume target.

12. The method of claim 9, wherein the purge volume is less than the first rinse volume.

13. A washing machine appliance comprising
a tub;
a basket positioned within the tub, the basket rotatable within the tub and defining a wash chamber for receipt of articles for washing;
a flow regulator;
a controller in operable communication with the flow regulator, the controller configured to execute a sanitation cycle comprising
determining a water fill volume target;
determining a first wash volume based on a fixed percentage of the water fill volume target, the first wash volume being less than the determined water fill volume target;
adding the first wash volume of water to the wash chamber;
initiating a secondary operation; and
adding a second wash volume of water, the sum of the first wash volume and second wash volume being substantially equal to the water fill volume target.

14. The washing machine appliance of claim 13, wherein the first wash volume is between about forty percent and about eighty percent of the water fill volume target.

15. The washing machine appliance of claim 13, wherein the secondary operation includes at least one of agitating the articles positioned within the wash chamber for a determined time period, or soaking the articles positioned within the wash chamber for a determined time period.

16. The washing machine appliance of claim 13, wherein adding the first wash volume of water to the wash chamber comprises adding the first wash volume of water substantially from a hot water tap of the flow regulator.

17. The washing machine appliance of claim 13, wherein the sanitation cycle further comprises
draining the first wash volume of water and the second wash volume of water from the wash chamber; and
rinsing the articles positioned within the wash chamber.

18. The washing machine appliance of claim 17, wherein rinsing the articles positioned within the wash chamber further comprises
adding a first rinse volume of water to the wash chamber;
draining a purge volume of water from the wash chamber; and
adding a second rinse volume of water to the wash chamber.

19. The washing machine appliance of claim 17, wherein the first rinse volume is less than the water fill volume target.

20. The washing machine appliance of claim 17, wherein the purge volume is less than the first rinse volume.
